(19) United States
(12) Patent Application Publication
(10) Pub. No.: US 2020/0357493 A1
(43) Pub. Date: Nov. 12, 2020

(54) MONITORING, CAPTURING, MEASURING AND ANNOTATING PHYSIOLOGICAL WAVEFORM DATA

(71) Applicant: CERNER INNOVATION, INC., KANSAS CITY, KS (US)

(72) Inventors: Lisa Kelly, Overland Park, KS (US); Judy Zakutny, Olathe, KS (US); Bradley Scott, Overland Park, KS (US)

(21) Appl. No.: 16/937,998

(22) Filed: Jul. 24, 2020

Related U.S. Application Data
(63) Continuation of application No. 13/094,652, filed on Apr. 26, 2011.

(51) Int. Cl. G16H 10/60

(52) U.S. Cl. CPC G16H 10/60

(57) ABSTRACT

Systems, methods, and computer-readable media for managing healthcare environments are provided. In embodiments, signals are received from more than one lead corresponding to a measurement associated with a patient. Real-time waveforms or physiologic data is displayed representing each signal. Events are detected for at least one of the waveforms or physiologic data. Temporary queues store the waveforms or physiologic data corresponding to the events, where they may be reviewed, measured, annotated, or saved to the patient's electronic medical record.

### CROSS REFERENCE TO RELATED APPLICATIONS

This application is a Continuation of claims priority to U.S. patent application Ser. No. 13/094,652, filed on Apr. 26, 2011, entitled “Monitoring, Capturing, Measuring and Annotating Physiological Waveform Data,” the entirety of the aforementioned application is incorporated by reference herein.

### BACKGROUND

Physiological waveform data is often used by clinicians to detect otherwise subtle changes or events that may be indicative of serious medical conditions. Devices associated with detecting these changes receive signals corresponding to measurements from leads connected to patients. These measurements are read continuously by the devices and are displayed against time as waveforms. In many instances, it is difficult for clinicians to review the physiological waveform data because the waveforms are displayed on or near the devices and reviewing real-time data is inconvenient and inefficient. In other instances, when an event is detected that needs to become part of the patient's medical record, paper strips of the waveforms are printed. Unfortunately, if these strips are lost or never make it into the medical record, clinicians cannot review historical physiologic data. A comprehensive solution is needed that allows clinicians to remotely access, annotate, measure, and save real-time and historical physiologic data directly to a patient's electronic medical record (EMR).

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Embodiments of the present invention relate to methods, systems and computer storage media having computer-executable instructions embodied thereon that, when executed, cause a computing device to perform a method of monitoring, capturing, measuring and annotating physiological waveform data. Signals are received from more than one lead corresponding to measurements associated with a patient. A waveform or physiologic data representing each signal is displayed. A manipulation of a time period associated with the display is received. An indication to record a first selected portion of the display and selected physiologic data to a temporary queue for a configurable period of time is received. An indication to save a second selected portion of the display and selected physiologic data to an electronic medical record associated with the patient is received. A third portion of the display and physiologic data is permanently deleted after the configurable period of time.

Embodiments of the present invention relate to methods, systems and computer storage media having computer-executable instructions embodied thereon that, when executed, cause a computing device to perform a method of monitoring, capturing, measuring and annotating physiological waveform data. Signals from more than one lead corresponding to measurements associated with a patient are received. A waveform or physiologic data representing each signal is displayed. An event in at least one of the waveforms is detected. A view of the event and corresponding data is provided. An event measurement of at least a portion of the event is received. An annotation for at least a portion of the event is received.

Embodiments of the present invention relate to methods, systems and computer storage media having computer-executable instructions embodied thereon that, when executed, cause a computing device to perform a method of monitoring, capturing, measuring and annotating physiological waveform data. A signal component receives signals from more than one device corresponding to measurements associated with a patient. A waveform or physiologic data representing each signal is displayed by a display component. An event component detects an event in at least one of the waveforms or physiologic data. The event and corresponding waveforms or data is received by a temporary queue component. The event and selected data is recorded in an EMR associated with the patient by a permanent save component.

### BRIEF DESCRIPTION OF THE DRAWINGS

Embodiments are described in detail below with reference to the attached drawing figures, wherein:

FIG. 1 is a block diagram of an exemplary computing environment suitable for use in implementing embodiments of the present invention;

FIG. 2 is an exemplary system architecture suitable for use m implementing embodiments of the present invention;

FIGS. 3-18 are illustrative screen displays in accordance with embodiments of the present invention; and

FIGS. 19-20 are flow diagrams of methods in accordance with an embodiment of the present invention.

### DETAILED DESCRIPTION OF THE INVENTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies.

Having briefly described embodiments of the present invention, an exemplary operating environment suitable for use in implementing embodiments of the present invention is described below.

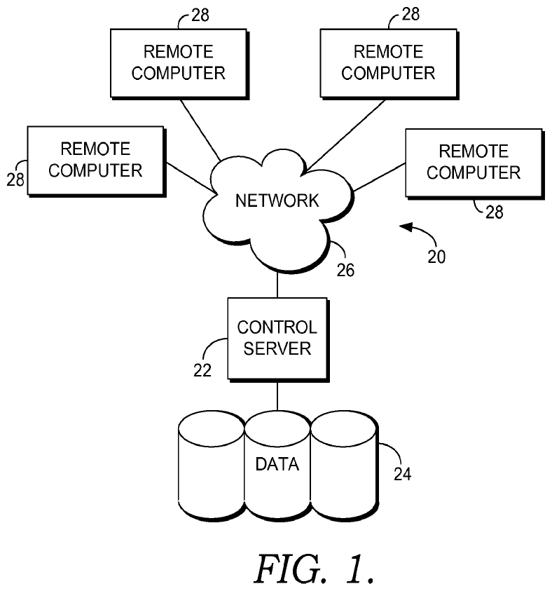

Referring to the drawings in general, and initially to FIG. 1 in particular, an exemplary computing system environment, a medical information computing system environment, with which embodiments of the present invention may be implemented is illustrated and designated generally as reference numeral 20. It will be understood and appreciated by those of ordinary skill in the art that the illustrated medical information computing system environment 20 is merely an example of one suitable computing environment and is not intended to suggest any limitation as to the scope of use or functionality of the invention. Neither should the medical information computing system environment 20 be interpreted as having any dependency or requirement relating to any single component or combination of components illustrated therein.

The present invention may be operational with numerous other general purpose or special purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, network PCs, minicomputers, mainframe computers, distributed computing environments that include any of the above-mentioned systems or devices, and the like.

The present invention may be described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in association with local and/or remote computer storage media including, by way of example only, memory storage devices.

With continued reference to FIG. 1, the exemplary medical information computing system environment 20 includes a general purpose computing device in the form of a control server 22. Components of the control server 22 may include, without limitation, a processing unit, internal system memory, and a suitable system bus for coupling various system components, including database cluster 24, with the control server 22. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus, and Peripheral Component Interconnect (PCI) bus, also known as Mezzanine bus.

The control server 22 typically includes therein, or has access to, a variety of computer-readable media, for instance, database cluster 24. Computer-readable media can be any available media that may be accessed by server 22, and includes volatile and nonvolatile media, as well as removable and non-removable media. By way of example, and not limitation, computer-readable media may include computer storage media. Computer storage media may include, without limitation, volatile and nonvolatile media, as well as removable and non-removable media implemented in any method or technology for storage of information, such as computer-readable instructions, data structures, program modules, or other data. In this regard, computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage, or other magnetic storage device, or any other medium which can be used to store the desired information and which may be accessed by the control server 22. By way of example, and not limitation, communication media includes wired media such as a wired network or direct-wired connection, and wireless media such as acoustic, RF, infrared, and other wireless media. Combinations of any of the above also may be included within the scope of computer-readable media.

The computer storage media discussed above and illustrated in FIG. 1, including database cluster 24, provide storage of computer-readable instructions, data structures, program modules, and other data for the control server 22. The control server 22 may operate in a computer network 26 using logical connections to one or more remote computers 28. Remote computers 28 may be located at a variety of locations in a medical or research environment, for example, but not limited to, clinical laboratories (e.g., molecular diagnostic laboratories), hospitals and other inpatient settings, veterinary environments, ambulatory settings, medical billing and financial offices, hospital administration settings, home health care environments, and clinicians' offices. Clinicians may include, but are not limited to, a treating physician or physicians, specialists such as intensivists, surgeons, radiologists, cardiologists, and oncologists, emergency medical technicians, physicians' assistants, nurse practitioners, nurses, nurses' aides, pharmacists, dieticians, microbiologists, laboratory experts, laboratory technologists, genetic counselors, researchers, veterinarians, students, and the like. The remote computers 28 may also be physically located in non-traditional medical care environments so that the entire health care community may be capable of integration on the network. The remote computers 28 may be personal computers, servers, routers, network PCs, peer devices, other common network nodes, or the like, and may include some or all of the elements described above in relation to the control server 22. The devices can be personal digital assistants or other like devices.

Exemplary computer networks 26 may include, without limitation, local area networks (LANs) and/or wide area networks (WANs). Such networking environments are commonplace in offices, enterprise-wide computer networks, intranets, and the Internet. When utilized in a WAN networking environment, the control server 22 may include a modem or other means for establishing communications over the WAN, such as the Internet. In a networked environment, program modules or portions thereof may be stored in association with the control server 22, the database cluster 24, or any of the remote computers 28. For example, and not by way of limitation, various application programs may reside on the memory associated with any one or more of the remote computers 28. It will be appreciated by those of ordinary skill in the art that the network connections shown are exemplary and other means of establishing a communications link between the computers (e.g., control server 22 and remote computers 28) may be utilized.

In operation, a clinician may enter commands and information into the control server 22 or convey the commands and information to the control server 22 via one or more of the remote computers 28 through input devices, such as a keyboard, a pointing device (commonly referred to as a mouse), a trackball, or a touch pad. Other input devices may include, without limitation, microphones, satellite dishes, scanners, or the like. Commands and information may also be sent directly from a remote healthcare device to the control server 22. In addition to a monitor, the control server 22 and/or remote computers 28 may include other peripheral output devices, such as speakers and a printer.

Although many other internal components of the control server 22 and the remote computers 28 are not shown, those of ordinary skill in the art will appreciate that such components and their interconnection are well known. Accordingly, additional details concerning the internal construction of the control server 22 and the remote computers 28 are not further disclosed herein.

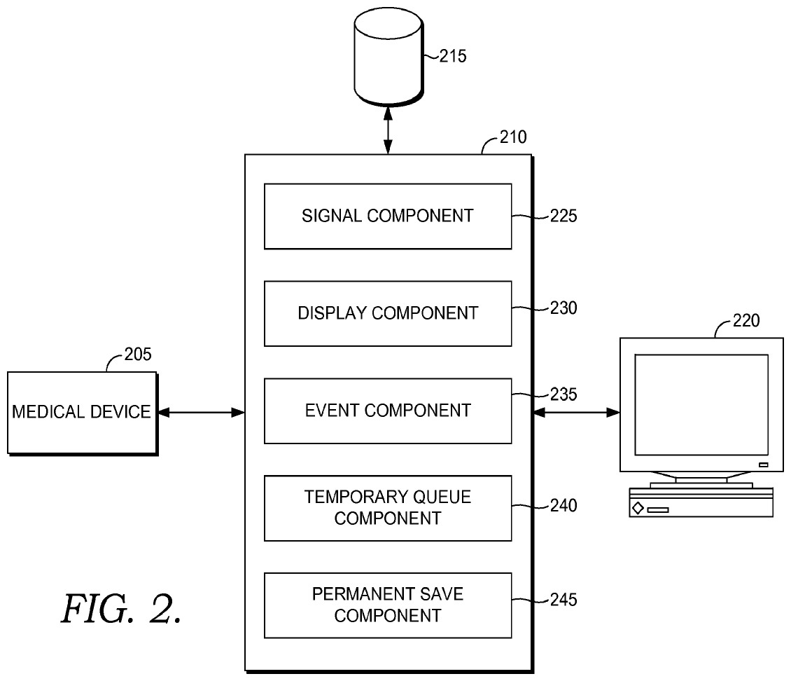

With reference to FIG. 2, a block diagram is illustrated that shows an exemplary computing system architecture for monitoring, capturing, measuring and annotating physiological waveforms. It will be appreciated that the computing system architecture shown in FIG. 2 is merely an example of one suitable computing system and is not intended as having any dependency or requirement related to any single module/component or combination of modules/components.

The computing system includes one or more medical devices 205, physiological waveform module 210, database 215 and graphical display 220. Physiologic data elements are received from device 205. A medical device 205 may be any device, stationary or otherwise, that may be used to treat a patient in a hospital, doctor's office, etc. For exemplary purposes only and not limitation, medical devices include cardiac monitors, cardiac output monitors, ICP monitors, ventilators, pumps (e.g., infusion pumps, balloon pumps), and the like.

Database 215 contains a variety of information data for the patient in a patient's electronic medical record (EMR). As utilized herein, the acronym “EMR” is not meant to be limiting, and may broadly refer to any or all aspects of the patient's medical record rendered in a digital format. Generally, the EMR is supported by systems configured to co-ordinate the storage and retrieval of individual records with the aid of computing devices. As such, a variety of types of healthcare-related information may be stored and accessed in this way. By way of example, the EMR may store one or more of the following types of information: patient demographic; medical history (e.g., examination and progress reports of health and illnesses); medicine and allergy lists/immunization status; laboratory test results, radiology images (e.g., X-rays, CTs, MRIs, etc.); evidence-based recommendations for specific medical conditions; a record of appointments and physician's notes; billing records; and data received from an associated medical device. Accordingly, systems that employ EMRs reduce medical errors, increase physician efficiency, and reduce costs, as well as promote standardization of healthcare. Graphical display device 220 may be a monitor, computer screen, project device or other hardware device for displaying output capable of displaying graphical user interfaces.

Physiological waveform module 210 receives and displays data from one or more medical devices for a patient. Physiological waveform module 210 may reside on one or more computing devices, such as, for example, the control server 22 described above with reference to FIG. 1. By way of example, the control server 22 includes a computer processor and may be a server, personal computer, desktop computer, laptop computer, handheld device, mobile device, consumer electronic device, or the like.

Physiological waveform module 210 comprises signal component 225, display component 230, event component 235, temporary queue component 240, and a permanent save component 245. In various embodiments, physiological waveform module 210 includes a historical queue component (not shown), a time manipulation component (not shown), a compressed view component (not shown), a drag and drop component (not shown), an event time line component (not shown), an annotation and measurement component (not shown), a signing component (not shown), and a purge component (not shown). Signal component 225, receives physiologic data from one or more medical devices 205. In various embodiments, signals associated with the physiologic data are received via leads. In various embodiments, the leads are internal electrodes, skin electrodes, or otherwise capable of measuring a signal or measurement associated with a patient. It will be appreciated that while physiological waveform module 210 is depicted as being connected to a single medical device 205, physiological waveform module 210 may receive physiologic data from multiple medical devices including medical devices monitoring multiple patients at multiple locations.

The data received by signal component 225 includes device related output from the medical device. For example, signal component 225 may receive data from cardiac monitors, cardiac output monitors, ICP monitors, ventilators, pumps (e.g., infusion pumps, balloon pumps), and the like. In one embodiment, the patient is continuously monitored and new data points are sent to the signal component 225 such that they may be plotted and displayed in a waveform quickly or in real-time. For clarity, real-time includes near real-time, taking into account latency or other typical delays between one or more devices communicating in a networked environment.

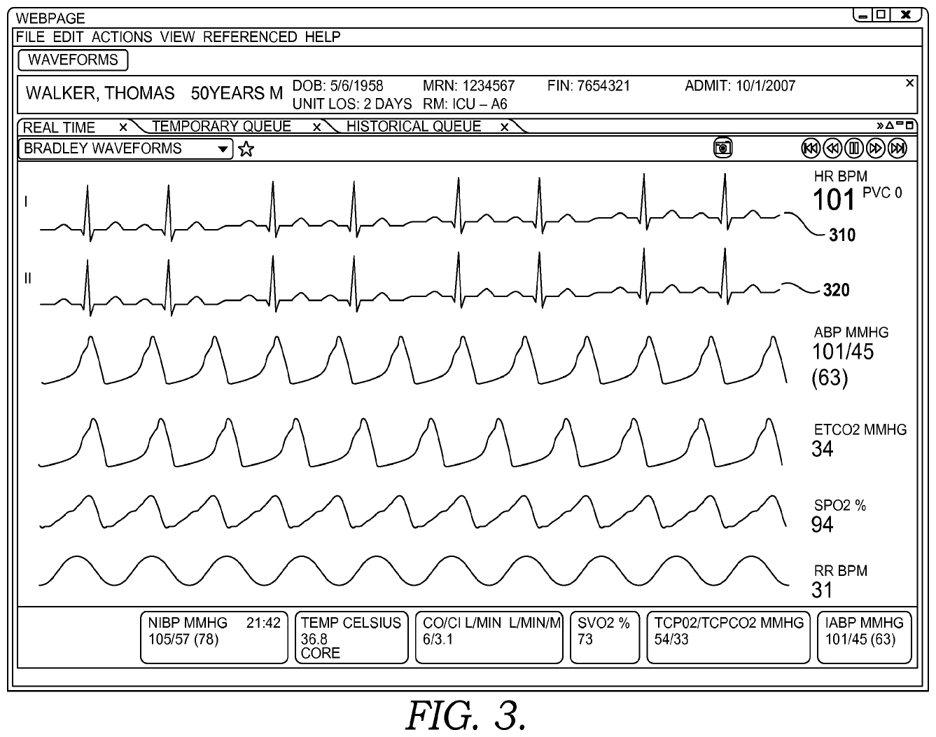

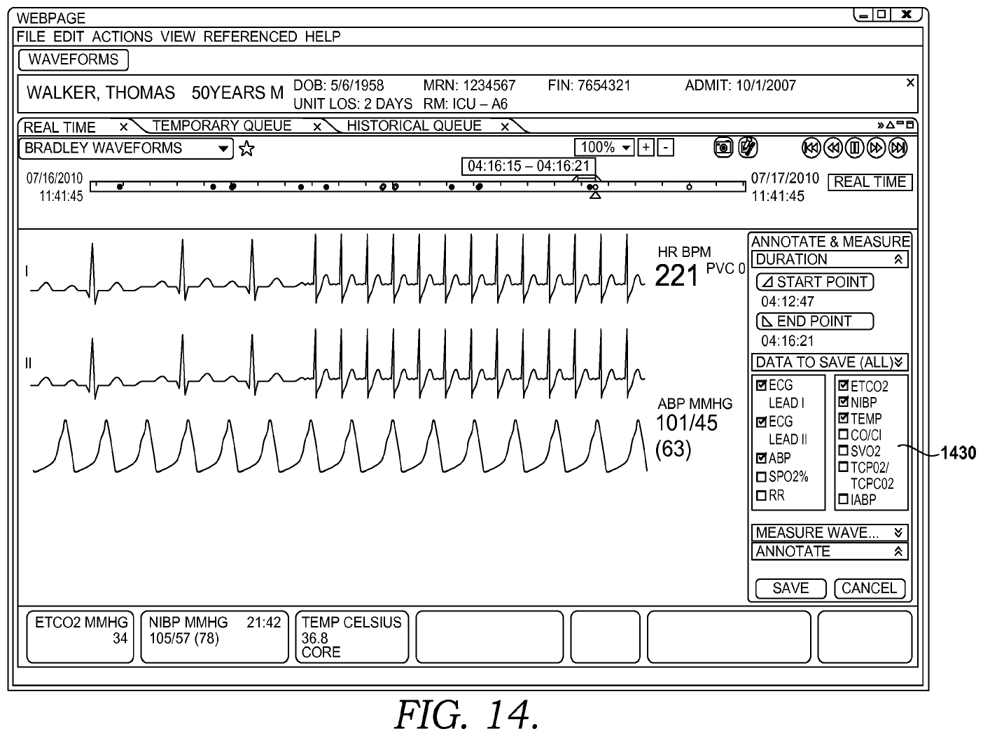

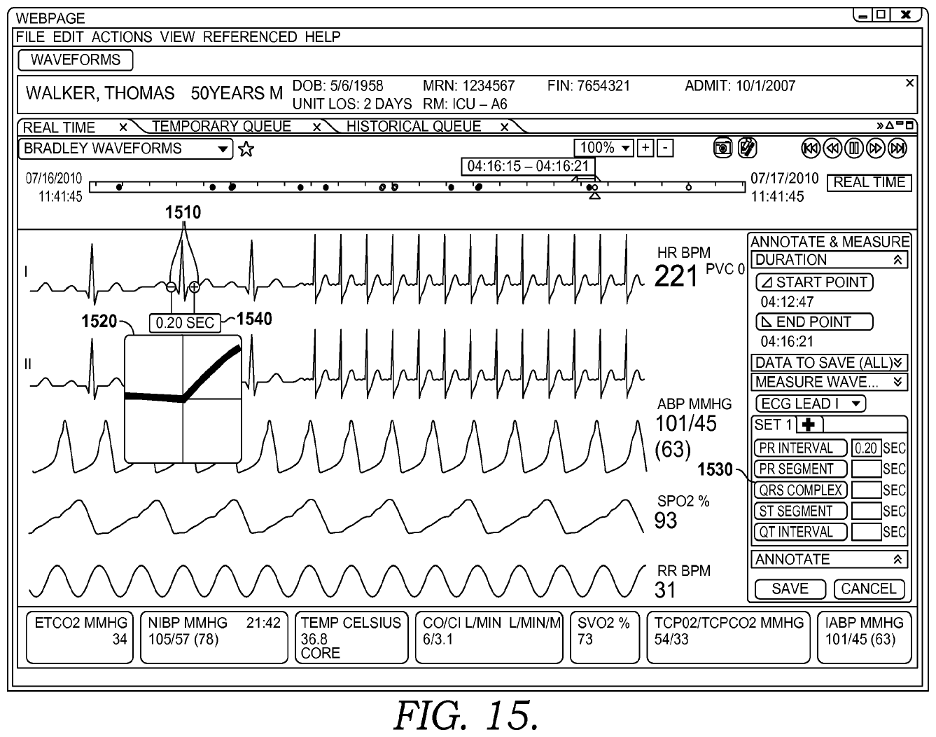

Referring now to FIG. 3, the signal component (225 in FIG. 2) receives signals from more than one device corresponding to measurements associated with a patient. A real-time component converts the data received from medical device 205 into electronic waveforms 310, 320 that can be displayed as tracings or graphs. In one embodiment, multiple waveforms 310, 320 are displayed of the same waveform type depending on how many leads are communicating signals to the signal component. The term waveform refers to the shape of a graph of the varying quantity against time. Exemplary electronic waveforms for data from medical devices are shown in FIGS. 3-15 and 17. For example, as data comes in indicating a patient's heart rate, it is graphed as a function of time in a waveform. In the exemplary waveforms, the newest data is plotted on the right side of the display. The prior data points are to the left of the newest plotted point. Exemplary data that is received and may be displayed in waveform includes, but is not limited to ECG, oxygen saturation (O2 sat), respiratory rate (RR), arterial blood pressure (arterial line), intra-aortic balloon pump (IABP), pulmonary artery blood pressure (PA), central venous pressure (CVP), intracranial pressure (ICP), carbon dioxide (end tidal CO2), ventilator waveforms, and the like. For exemplary purposes only and not limitation, physiologic data includes noninvasive blood pressure (NIBP), temperature, cardiac output/cardiac index (CO/CI), saturated venous oxygen (SvO2), transcutaneous CO2/O2 (tcpO2/tcpCO2), ventilator data, and the like.

In one embodiment, the real-time display component (230 in FIG. 2) provides a clinician with a configurable time period of real-time waveforms and physiologic data values. For example, a clinician may require a display that contains waveforms and physiologic data for the last twenty-four hours. In addition to providing a real-time display of the waveforms and physiologic data, the real-time component provides, in this example, the last twenty-four hours of waveforms and physiologic data.

In one embodiment, an event component (235 in FIG. 2) detects an event in at least one of the waveforms or physiologic data. The event is based on thresholds or parameters that allow the events to be automatically saved rather than requiring a clinician to manually save each event. Once an event is detected for which a threshold or parameter is met or exceeded, the waveforms and data corresponding to the event is communicated to the temporary queue component.

In one embodiment, a temporary queue component (240 in FIG. 2) receives the waveforms or data corresponding to the event automatically from the event component based on the thresholds or parameters. In one embodiment, a portion of time prior to the actual event is saved in association with the waveforms or data corresponding to the event. This allows a clinician to review what was going on with the patient (i.e., waveforms and data) immediately prior to the event. In another embodiment, the temporary queue component allows a clinician to manually and temporarily save events to the temporary queue. For example, a clinician may desire to temporarily save an event for later review or documentation.

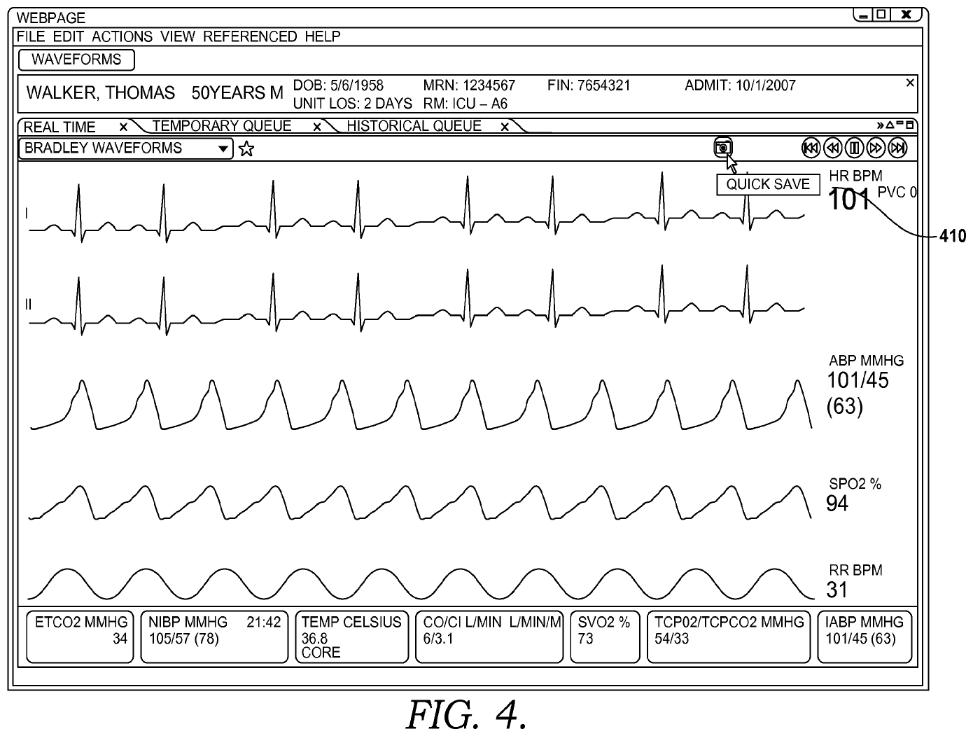

Referring to FIG. 4, a button, such as a quick save button 410, may be selected by the clinician to save the current display. A clinician may review temporarily saved events in the temporary queue until they are permanently saved or purged. In various embodiments, the temporary queue is comprised of various sections.

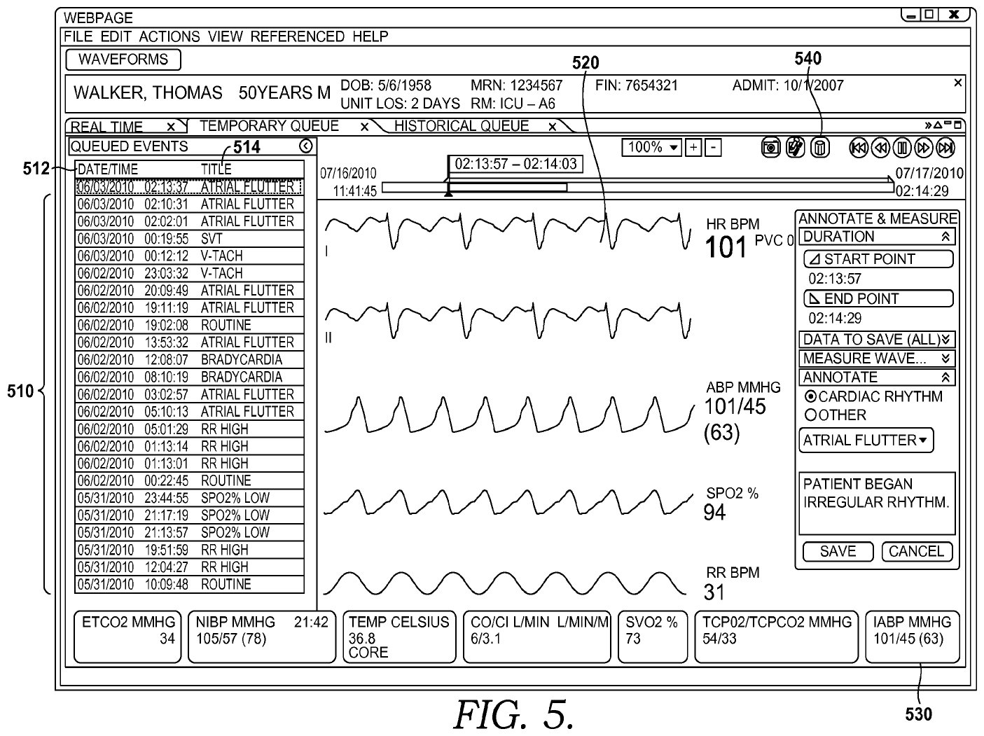

Referring to FIG. 5, in one embodiment, a queued events section 510 displays all of the temporarily saved waveforms. In yet another embodiment, a waveform section 520 contains waveforms and their associated physiologic data values and the physiologic data section 530 contains the physiologic data values. The queued events section 510 lists the temporarily saved waveforms. In one embodiment, this section can be collapsed and re-expanded horizontally. Columns in the queued events section 510 include a date/time column 512 and a title column 514. The date/time column 512 shows the start date and time for the event that was saved. The title column 514 displays the title of the event that was saved. Details for each event may be displayed, in one embodiment, as the cursor hovers over the event. In one embodiment, a clinician can delete temporarily saved waveforms by clicking a delete button 540. Inadvertently saved or unnecessary waveforms are then removed from the temporary queue.

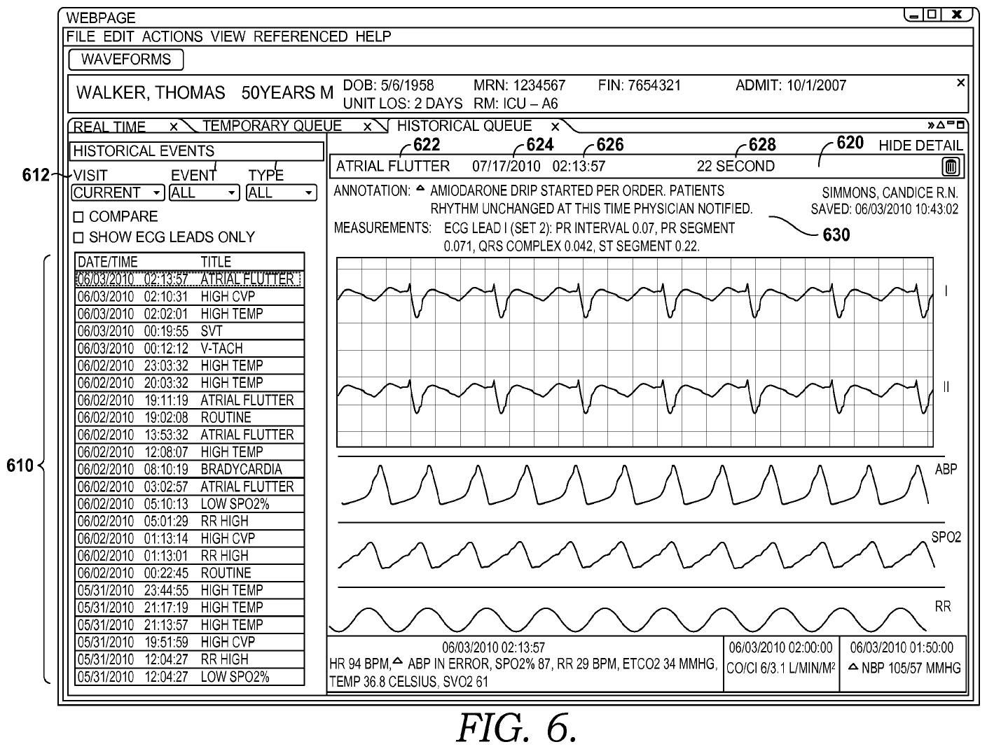

Referring now to FIG. 6, in one embodiment, a historical queue component (not shown in FIG. 2) allows a clinician to view permanently saved waveforms and physiologic data values from a central location. Events 610 that have been signed are permanently stored in the historical queue. For example, in one embodiment, a waveform and physiologic data may need to be reviewed and signed by a clinician. Once the waveform and physiologic data has been signed, it is stored permanently in the historical queue. In various embodiments, events may be filtered by visit, event category, and event type. A title bar 620 displays the title 622 of the selected event, the event start date 624 and time 626, and the duration 628 of the event. In one embodiment, this section can be collapsed and re-expanded horizontally. The title bar remains stationary, in one embodiment, while a clinician scrolls vertically or horizontally through the event. In one embodiment, a details section 630 displays associated annotations, measurements, the name of the clinician that signed the event, the signed date/time, or a combination thereof. In one embodiment, events that have been modified have a special indication, such as a blue triangle next to the modification. For example, if an annotation for an event was modified, a blue triangle would appear in the historical queue next to that modified portion of the event.

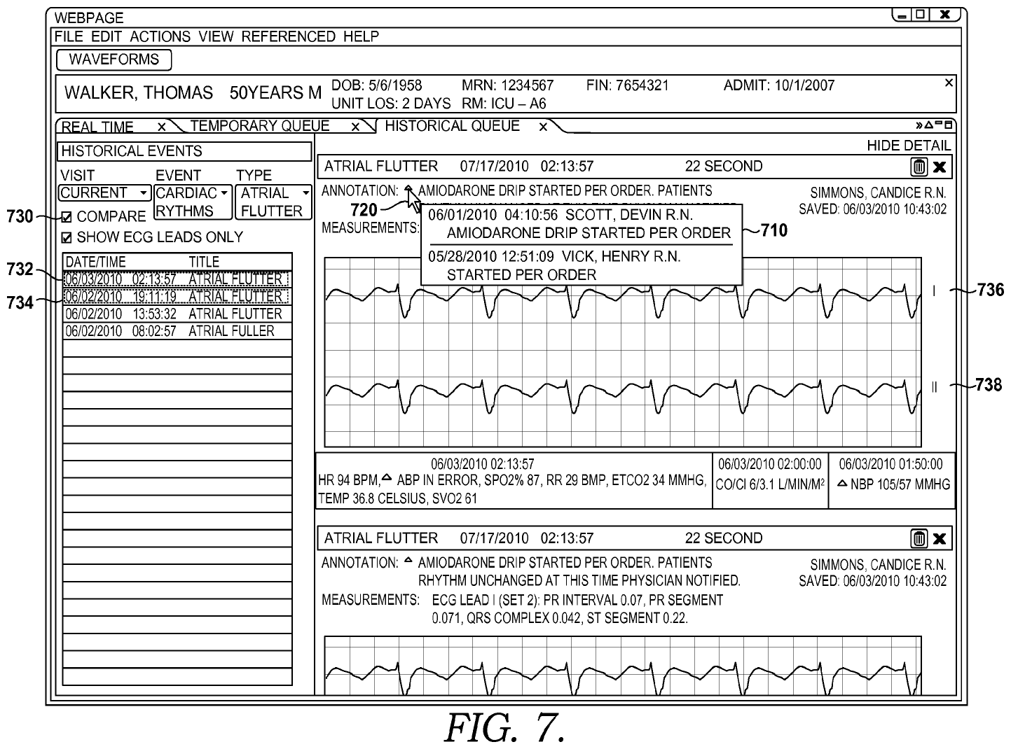

Referring to FIG. 7, in one embodiment, a clinician can view details 710 related to an event merely by positioning the cursor 710 over the event. If the text cannot be fully displayed for annotations, measurements, or both, cursor can be positioned, in one embodiment, over the annotation and measurements section and all of the annotations and measurements are displayed. In one embodiment, a compare option 730 allows a clinician to compare the waveforms between multiple saved events 732, 734 at the same time. In another embodiment, a show ECG leads only button allows a clinician to view waveforms to ECG leads only 736, 738.

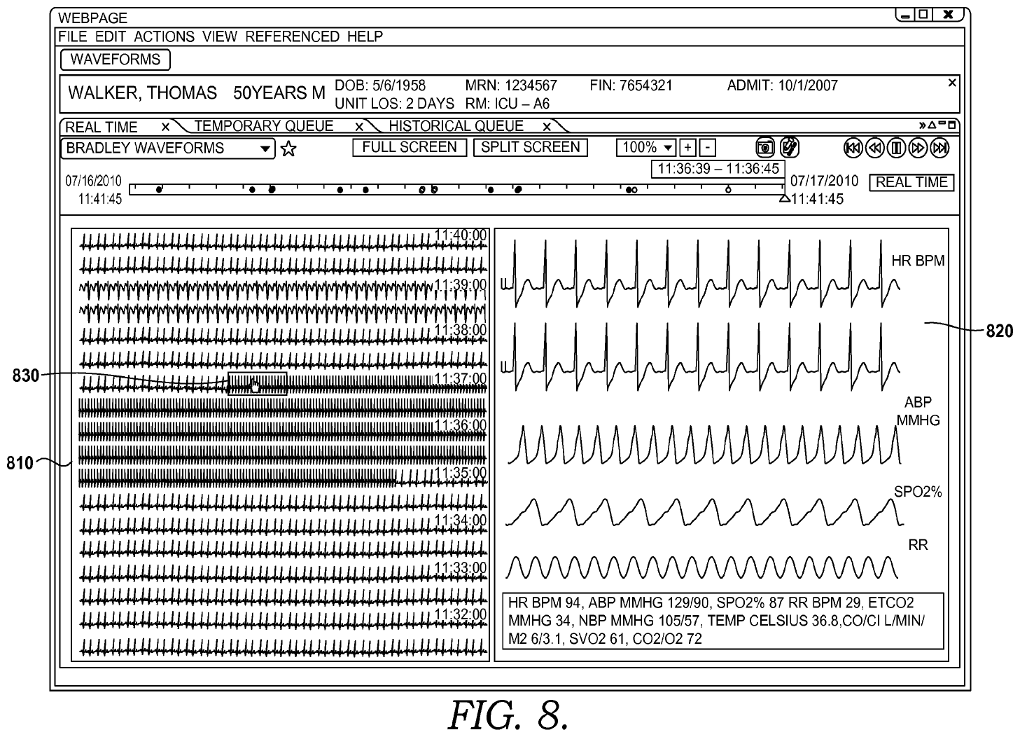

Referring to FIG. 8, in one embodiment, a compressed view component provides a compressed view of an extended time period of waveform data. For example, the left side of the display provides a compressed view 810 of twenty-four hours of waveform data. The right side of the display 820 is the actual portion of the waveform and associated data for the compressed portion of the waveform identified within the box 830 on the left side of the display.

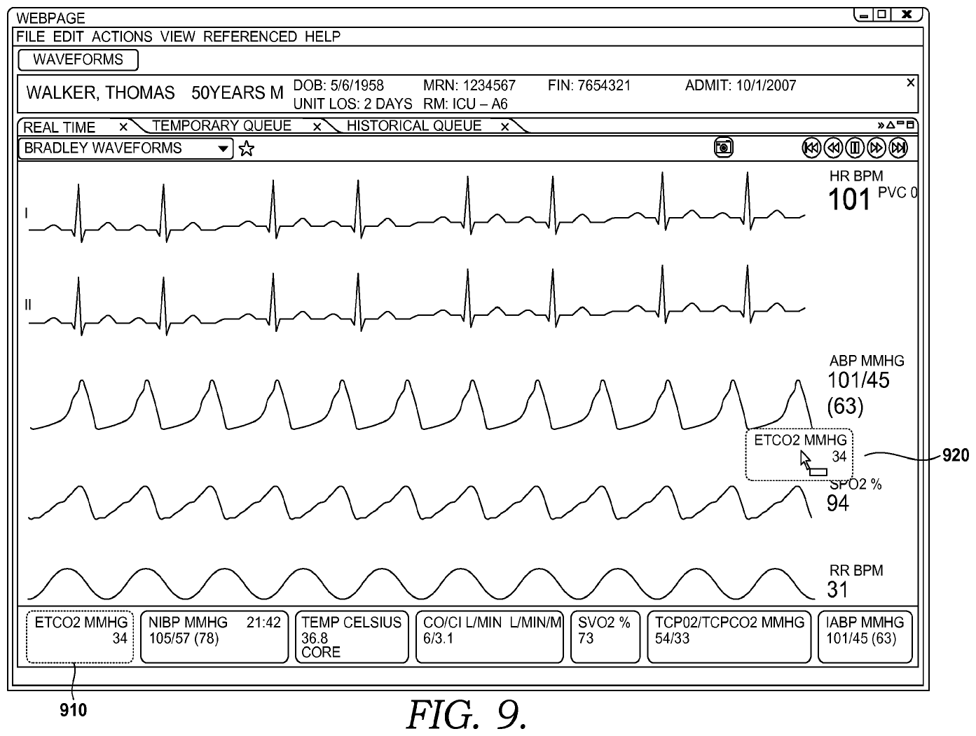

Referring to FIG. 9, in one embodiment, a drag and drop component allows a clinician to rearrange the data into a custom layout. In various embodiments, a drag and drop option can be accessed in the real-time display, the paused display, and the temporary queue. For example, if a clinician desires to reorder waveforms representing different data, the clinician merely needs to drag a waveform to the desired location within the waveform section of the display. If the clinician desires to see the waveform associated with a particular aspect of physiologic data 910, the clinician merely needs to drag that component 920 into the waveform section of the display and its waveform is displayed. If the clinician no longer desires to see a waveform associated with a particular lead, the clinician merely needs to drag that waveform into the physiologic data section of the display and that particular aspect of physiologic data is displayed.

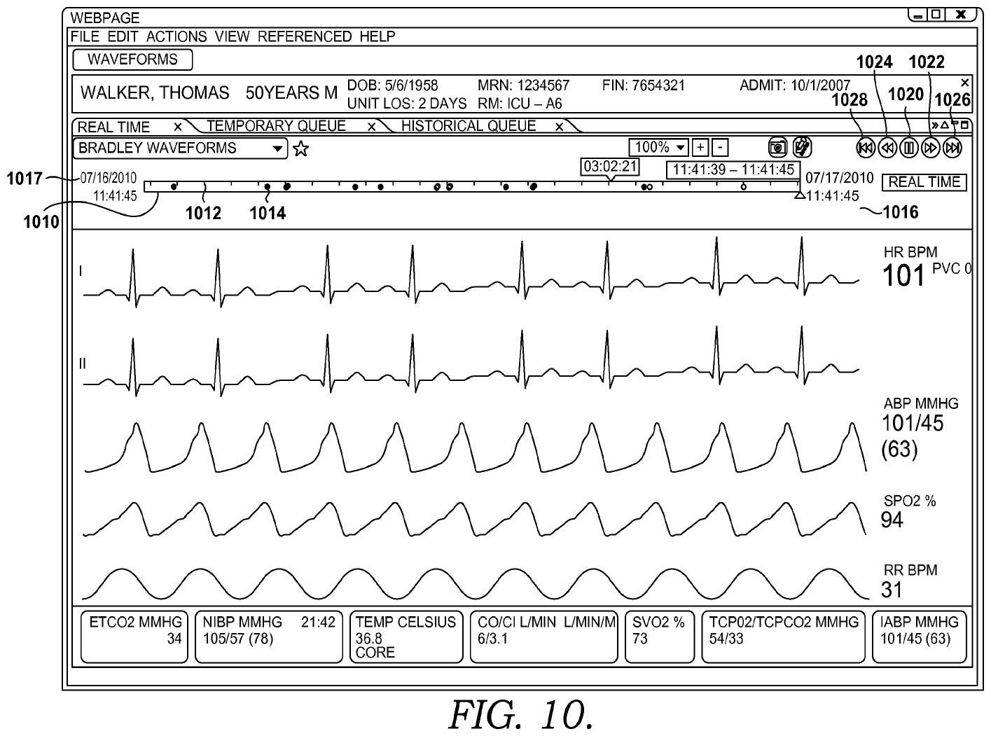

Referring now to FIG. 10, an event time line component (not shown in FIG. 2) provides, in one embodiment, tools allowing a clinician to view the waveforms more accurately. The event time line 1010 provides information related to the date and time associated with aspects of the various waveforms. In various embodiments, the event time line 1010 can be accessed in the paused display and the temporary queue. For example, the event time line 1010 displays tick marks on the time line representing hour increments. If the clinician needs to verify the time an event occurred in a waveform, the clinician merely needs to position the cursor over the time line and the corresponding time is displayed. In addition, events 1014 that have been saved permanently by the permanent save component are displayed on the event time line for the configurable time period (as described above). A date and time label is displayed at the beginning and end of the event time line. The date and time on the right-side of the display 1016 represents the date and time the pause occurred. The date and time on the left-side of the display 1017 represents the date and time for the configurable time period prior to the time the pause occurred. In various embodiments, additional markings are displayed for each event on the timeline. For example, a clear circle on the time line may represent an event without annotations and measurements. A filled circle may represent an event that has annotations and/or measurements. A total of three icons stacked may represent when more than one saved event exists and the times overlap.

Still referring to FIG. 10, a time manipulation component (not shown in FIG. 2) allows a clinician to view the waveforms more accurately by pausing the real-time display, such as by clicking a pause button 1020. In various embodiments, the time manipulation component can be accessed in the real-time display and the temporary queue. After clicking the pause button, the display is changed to an event time line based on the time the real-time display was paused. Continuing with the above example, if the configurable time period is set to twenty-four hours, then the paused view component will show an event time line for the last twenty-four hours. Additional control buttons are provided to the clinician by the time manipulation component that allow the clinician, in various embodiments, to scroll through, skip forward, via a skip forward button 1026, skip backwards, via a skip backwards button 1028, play, rewind, via a rewind button 1024, or fast-forward, via a fast forward button 1022, the waveform and increase or decrease the zoom percentage of a selected waveform. If the clinician selects to play the paused waveform, the waveforms play until the originally paused time is reached. If selecting to fast-forward through the paused waveforms, the clinician may select a desired speed to fast-forward through the waveforms until the originally paused time is reached. A clinician may also choose to scroll horizontally through the paused waveforms to review different sections of a waveform. If selecting to rewind through the paused waveforms, the clinician may select a desired speed to rewind through the waveforms until the beginning of available data is reached. Once the clinician is ready to return to the real-time display, such as by clicking a real-time button, the real-time component resumes the display to the moment in time when the real-time button was clicked and a real-time display is provided.

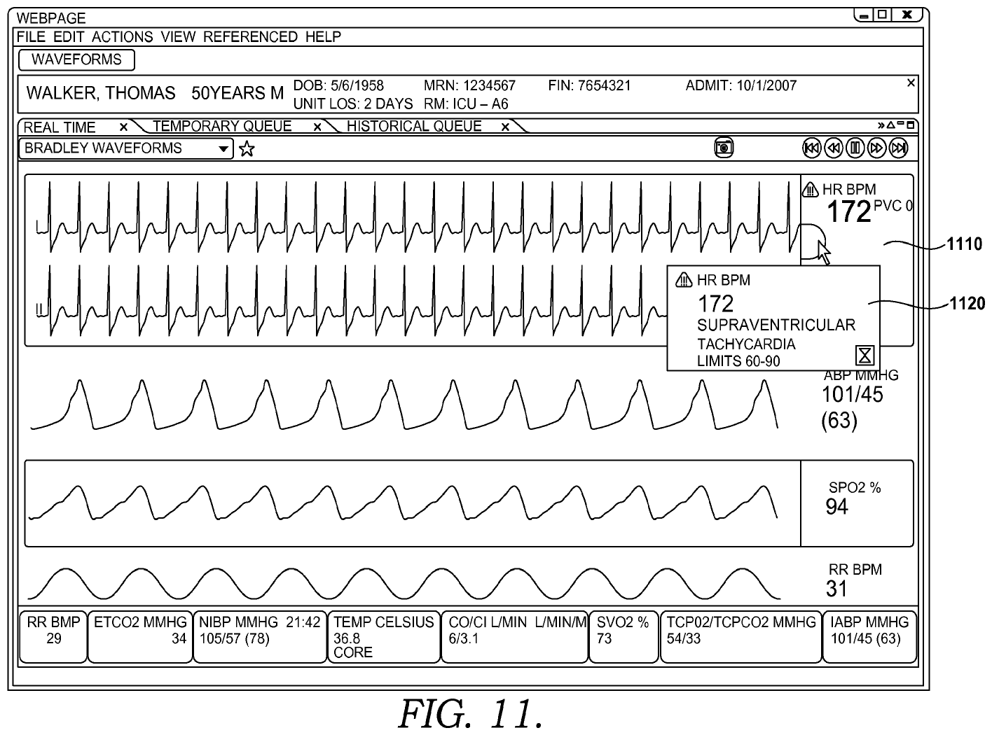

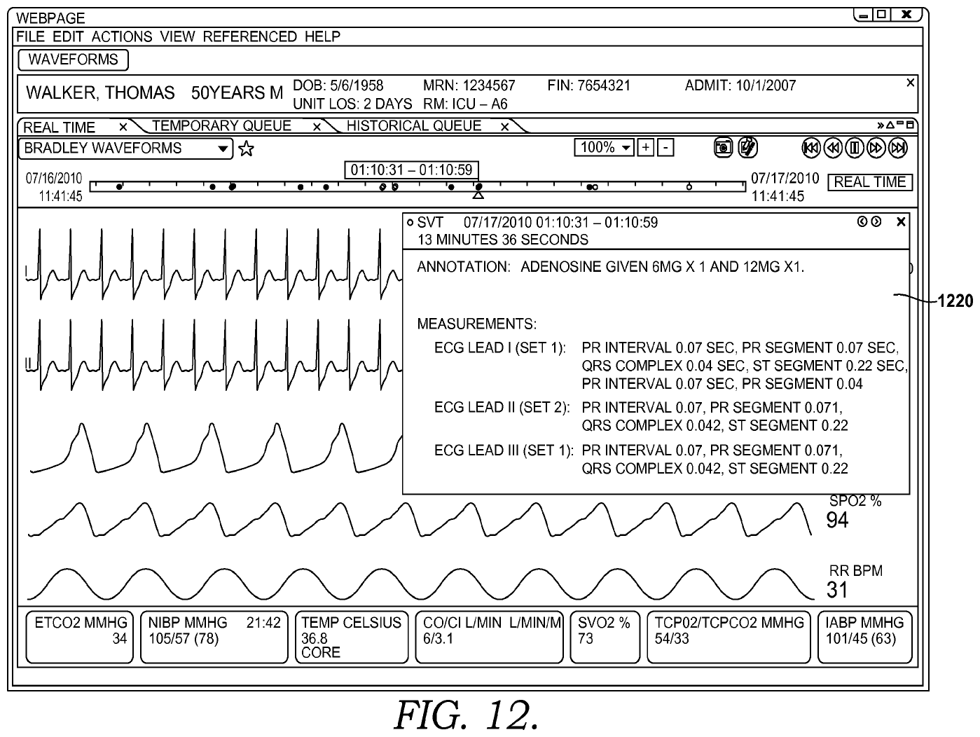

Referring now to FIGS. 11 and 12, in one embodiment, if the clinician desires to view the details related to an event, the clinician positions the cursor 1110 over the physiologic data and information 1120 corresponding to an alert. In one embodiment, alert information related to the alerts is displayed. In one embodiment, the alert information includes a severity icon, title of the alert, alert limits, and an icon to launch an alerts limit option. The alerts limit option, in one embodiment, allows a clinician to configure alert limits and thresholds.

Referring now to FIG. 12, in one embodiment, event summary 1220 is displayed when selecting an event on the timeline. The event summary includes, in one embodiment, an event title, an event date, an event start and end time, and event duration. In one embodiment, if the clinician positions the cursor over multiple markings, the event title, event date, event start and end time is displayed from newest to oldest based on the event start date and time. The event summary may include an event start date, an event start and end time, associated annotations (if they exist), and associated measurements (if they exist). In one embodiment, the clinician may skip forward or skip backwards through the various event summaries available on the event time line by clicking the appropriate arrow in the event summary dialogue box.

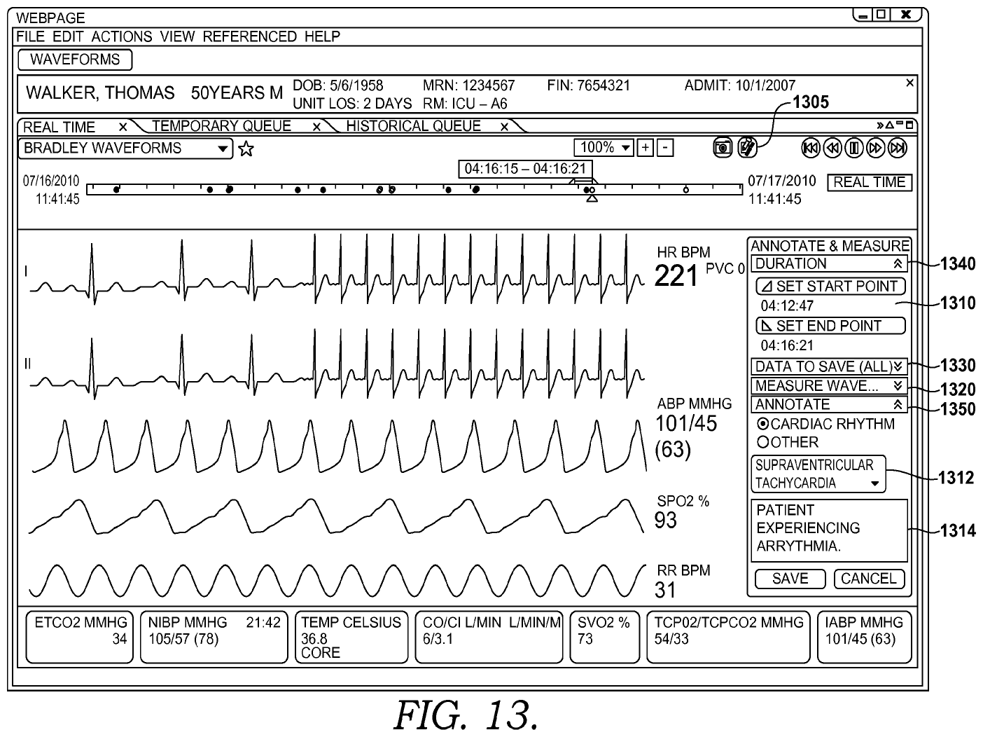

Referring now to FIGS. 13-15, in one embodiment, an annotation and measurement component (not shown in FIG. 2) receives input from a clinician relevant to the waveforms or data. A dialog box 1310 allows the clinician to add annotations 1350 and measurements 1320 or set the duration of the event to be saved. In various embodiments, the annotation and measurement component can be accessed in the paused display and the temporary queue. In various embodiments, the dialog box further includes a data to save section 1330, 1430 that allows a clinician to select and deselect individual waveforms and physiologic data values. While the display is paused, the clinician may select an annotate and measure button 1305 to access the annotate and measure dialog box. In one embodiment, while the annotate and measure dialog box 1310 is open, hash marks are displayed on the portions of the waveform section that are outside the start and end times defined in the duration section. In one embodiment, distinguishing characteristics are used to denote the start and end times of the event. For example, a green vertical line, in one embodiment, represents start point of the waveforms and a red vertical line represents the end point of the duration. Once the duration is set via the dialog box 1340, the display is updated to display the beginning and end times of the waveform displays. In one embodiment, a distinguishing characteristic, such as a blue duration bar, is displayed between the start and end points to illustrate the duration of the event.

In one embodiment, a measure waveforms section allows a clinician to create measurements on ECG waveforms. Such measurements allow a clinician to assess a patient's condition with greater accuracy. When a measurement is desired, the clinician selects the annotate and measure button, in one embodiment, and selects measure waveforms. Calipers 1510 and grid lines are displayed on a selected ECG lead. A magnifying glass, in one embodiment, appears when a clinician clicks on a cross hair circle portion of the caliper arm. This provides a magnified view 1520 of the portion of the waveforms the caliper cross hair is centered on. In one embodiment, the calipers can be dragged to the desired portion of the waveform. The width of the calipers, corresponding to a time unit of the waveform, may be adjusted as desired. Once the clinician has set the calipers to the desired portion of the waveform, one or more sets of measurements may be selected. In various embodiments, buttons 1530 for measuring PR Interval, PR Segment, QRS Complex, ST Segment, QT Interval are displayed. When a button is selected, the appropriate numerical measurement value 1540 for the selected caliper measurement is displayed. In one embodiment, multiple sets of measurements may be selected per lead. The unit of measure displayed for each of the available caliper measurements is configurable, in one embodiment.

Referring back to FIG. 13, in one embodiment, an annotate section allows a clinician to add titles 1312 and annotations 1314 to events the clinician desires to save. For example, a clinician may desire to specify the type of event. In one embodiment, the clinician selects between a cardiac rhythm event type or an other event type. In one embodiment, upon selecting a cardiac rhythm event type, a drop down list of various cardiac rhythm event types is displayed for selection as the event title. Cardiac rhythm event types include accelerated idioventricular rhythm (AIVR), arrhythmias suspended, asystole, atrial escape complex, atrial escape rhythm, atrial fibrillation, atrial flutter, atrial tachycardia, bigeminy, bradycardia, bundle branch block, couplet alarm, first degree heart block, high PVC, irregular arrhythmia, junctional escape rhythm, junctional tacahycardia, left bundle branch block, low PVC, multifocal atrial tachycardia, normal sinus rhythm, paced, pause, PVC, R On T, reentrant tachycardia, right bundle branch block, second degree heart block type I, second degree heart block type II, sinus arrest, sinus arrhythmia, sinus bradycardia, sinus pause, sinus tachycardia, ST alarm, ST AVF alarm, ST AVL alarm, AT AVR alarm, ST high, ST I alarm, ST II alarm, ST III alarm, ST LO, ST VI alarm, ST V2 alarm, supraventricular tachycardia, supraventricular tachycardia with aberration, tachycardia, third degree heart block, torsades de pointe, trigeminy, V-FibN-Tach, ventricular bradycardia, ventricular escape rhythm, ventricular fibrillation, ventricular tachycardia, wandering atrial pacemaker, and wide ORS tachycardia unknown origin.

In one embodiment, upon selecting an other event type, a drop down list of various other event types is displayed for selection as the event title. Other event types include ABP sensor disconnected, apnea, device association, high CVP, high diastolic NIBP, high diastolic CO2, high inspired CO2, high mean ABP, high mean NIBP, high mean PA, high O2 concentration, high RR, high SPO2, high systolic ABP, high systolic NIBP, high systolic PA, high temperature, lead failure, low CO2, low CVP, low diastolic ABP, low diastolic NIBP, low diastolic PA, low expired CO2, low inspired CO2, low mean ABP, low mean NIBP, low mean PA, low O2 concentration, low RR, low diastolic PA, low expired CO2, low inspired CO2, low mean ABP, low mean NIBP, low mean PA, low O2 concentration, low RR, low SpO2, low systolic ABP, low systolic NIBP, low systolic PA, low temp, no ECG signal, probe is not connected, probe off patient, and scheduled.

An annotate box section 1314 is displayed, in one embodiment, allowing a clinician to optionally comment on the event being saved. After an event title 1312 is selected and the clinician has determined whether additional comments are necessary, a sign button is enabled, in one embodiment, by a signing component (not shown in FIG. 2). Once the sign button is selected, the signing component communicates with the permanent save component indicating that a clinician has signed a record corresponding to the event.

The permanent save component (245 in FIG. 2) saves the selected waveforms or data corresponding to the event as an image that can be viewed from the historical queue or other clinical applications. Events that are thirty seconds or less in length, in one embodiment, has one set of physiologic data values saved. Events greater than thirty seconds in length, in one embodiment, have two sets of physiologic data values saved, one corresponding to the start time of the event and one corresponding to the end time of the event.

In one embodiment, a purge component (not shown in FIG. 2) purges waveforms and data from the temporary queue that has not been permanently saved by the permanent save component. The purge is based on configurable parameters for age and frequency. When the parameters are met, the system purges the temporarily waveforms and data automatically. For example, if it is desired to retain waveforms and data in the temporary queue for twenty-four hours, the purge age threshold is set to twenty-four hours. After a set of waveforms and data exceeds twenty-four hours in the temporary queue, they are automatically purged from the system at the next scheduled purge process and are no longer available to a clinician. The purge frequency parameter controls how often the purge process runs within the system. For example, if the purge frequency parameter is set to twelve hours, then the purge process will run every twelve hours and will purge from the system any waveform and data that has exceeded the purge age threshold.

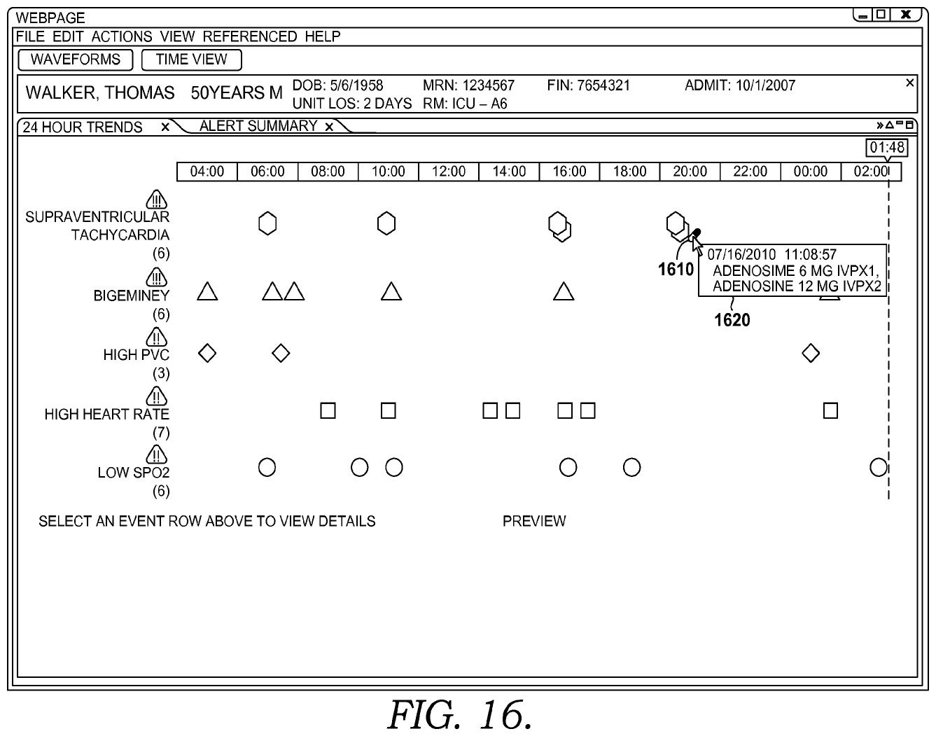

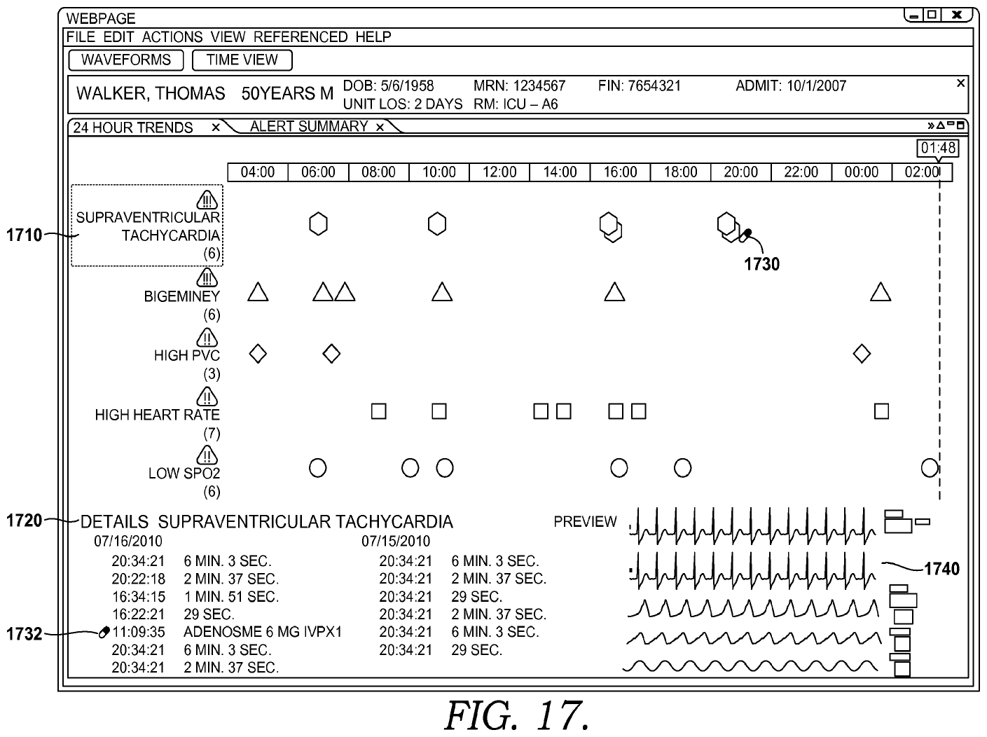

Referring now to FIGS. 16-18, illustrative screen displays provide patient summary data for review by clinicians in accordance with embodiments of the present invention. Referring specifically to FIG. 16, twenty-four hour trends are displayed for a patient. A clinician can move the cursor over an event 1610 on the timeline and details are provided in hover box 1620. Referring now to FIG. 17, a clinician may desire to review summary data for all events within a specific category. For example, if the clinician selects “Supraventricular Tachycardia” 1710, a detail summary for the six events 1720 that occurred for that category are displayed. A medication indicator 1730 on the timeline is provided indicating that a medication was administered to the patient. Medication details 1732 are provided in the details section, in addition to the events. A preview 1740 of available waveforms is also displayed in the preview section.

In one embodiment, a unit component (not shown in FIG. 2) provides a view of waveforms and physiologic data for multiple patients. In one embodiment, the unit view comprises a view of waveforms and physiologic data for each patient assigned to a clinician. In one embodiment, the unit view comprises a view of waveforms and physiologic data for a unit within a medical facility. The unit view allows a clinician to select the view for a single patient and access any of the components described herein. The unit view further allows a clinician to customize a view for any number of patients according to clinician preferences.

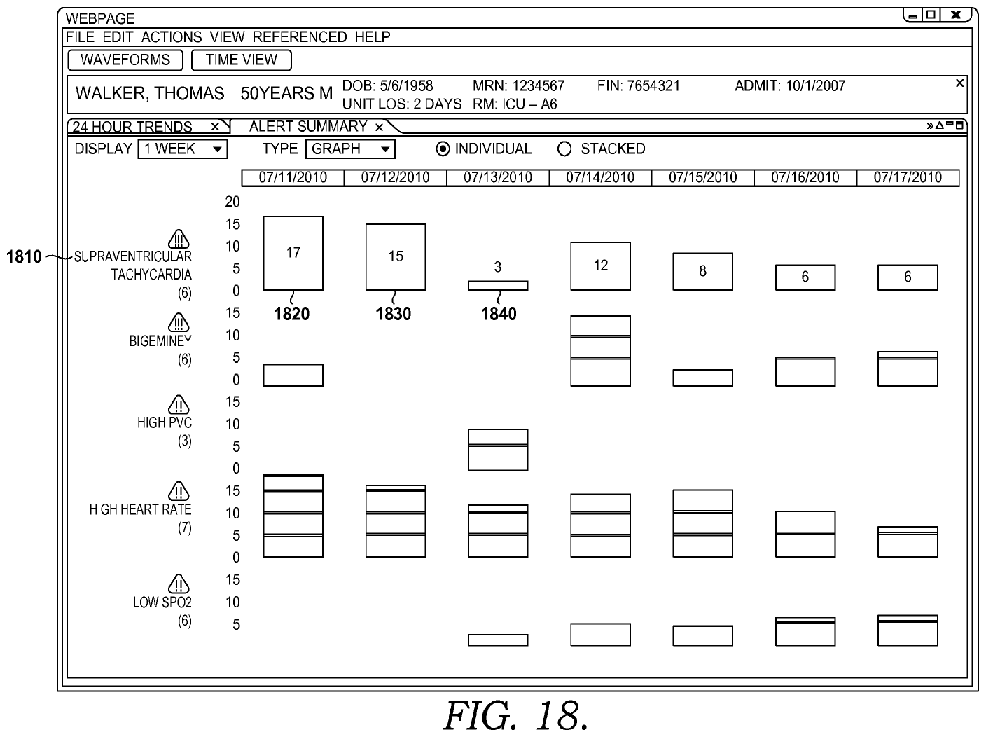

Referring now to FIG. 18, a graph summary displays a total number of events for a patient over a configured period of time. For example, the clinician is able to quickly review “Supraventricular Tachycardia” events 1810 for a patient over a one week period. The clinician is able to determine that the patient had 17 events 1820 on Jul. 11, 2010, 15 events 1830 on Jul. 12, 2010, and 3 events 1840 on Jul. 13, 2010. Such information may provide a clinician insight into conditions or treatments that can alleviate the patient's symptoms.

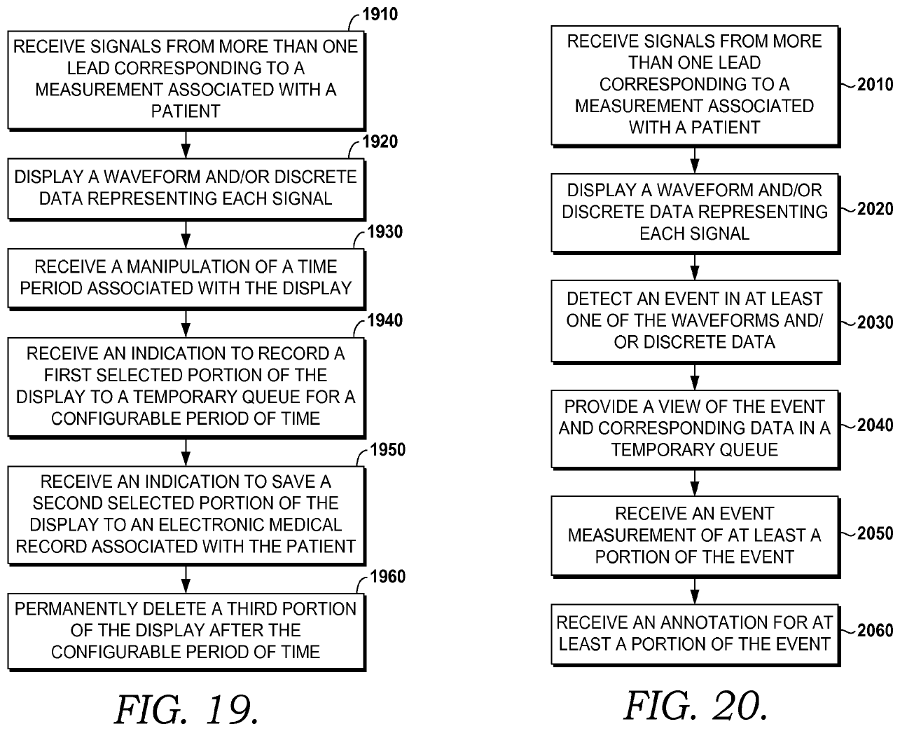

Referring now to FIG. 19, an illustrative flow diagram 1900 is shown of a method for capturing physiological data. At step 1910, signals from more than one lead corresponding to a measurement associated with a patient are received. A waveform and/or physiologic data representing each signal is displayed at step 1920. In one embodiment, an indication to display dragged physiologic data is received. In one embodiment, an indication to display dragged waveforms as additional physiologic data is received. For example, the clinician may determine that a waveform will better assist the clinician in determining whether an event is occurring. The clinician can drag the concerning component of physiologic data into the waveform section of the display, and if that particular component of physiologic data is capable of being displayed as a waveform, its waveform will be displayed. On the other hand, if the clinician determines that a particular waveform is not necessary and can be just as informative in the physiologic data display, the clinician may drag the unnecessary waveform into the physiologic data section and only the data will be displayed.

A manipulation of a time period associated with the display, at step 1930, is received. In one embodiment, the manipulation of a time period associated with the display includes playing, panning, scrolling, pausing, rewinding, or fast-forwarding the display.

At step 1940, an indication to record a first selected portion of the display to a temporary queue for a configurable period of time is received. In one embodiment, an indication to display the first selected portion of the display is received. For example, the clinician may desire to review the first selected portion. Such review may initially occur in the real-time display or in the temporary queue. The clinician may further desire to measure at least a portion of the first selected portion. In one embodiment, an event measurement associated with at least a portion of the first selected portion of the display is received. It may also be desirable for the clinician to annotate the event for documentation purposes. In one embodiment, an annotation associated with at least a portion of the first selected portion of the display is received.

An indication to save a second selected portion of the display to an electronic medical record associated with the patient is received at step 1950. A third portion of the display is permanently deleted, at step 1960, after the configurable period of time.

Referring now to FIG. 20, an illustrative flow diagram 2000 is shown of a method for measuring and annotating physiological data. At step 2010, signals from more than one lead corresponding to a measurement associated with a patient are received. A waveform and/or physiologic data representing each signal is displayed at step 2020. An event in at least one of the waveforms and/or physiologic data is detected at step 2030. At step 2040, a view of the event is provided. An event measurement, at step 2050, of at least a portion of the event is received. At step 2060, an annotation for at least a portion of the event is received.

In one embodiment, an adjustment of time associated with the display is received. In various embodiments, the adjustment of time includes rewinding, fast forwarding, pausing, or any combination thereof.

In one embodiment, the view of the event is stored in a temporary queue for a configurable period of time. This view facilitates a clinician's review of the event. In one embodiment, the view of the event is purged from the temporary queue after the configurable period of time.

In one embodiment, an indication form a clinician to sign a selected view is received. Such an indication may be received after the clinician has reviewed, annotated, and/or measured the selected view. Once the selected view is signed, the selected view is saved, in one embodiment, to an electronic medical record associated with the patient. In one embodiment, the saved event is displayed in a historical queue. For example, supposed a clinician desires to review the medical history of a patient. If any saved events exist in the patient's EMR, they may be reviewed in the historical queue. In one embodiment, waveforms and physiologic data that have been permanently saved may be reviewed by a clinician within the EMR.

Many different arrangements of the various components depicted, as well as components not shown, are possible without departing from the scope of the claims below. Embodiments of our technology have been described with the intent to be illustrative rather than restrictive. Alternative embodiments will become apparent to readers of this disclosure after and because of reading it. Alternative means of implementing the aforementioned can be completed without departing from the scope of the claims below. Certain features and subcombinations are of utility and may be employed without reference to other features and subcombinations and are contemplated within the scope of the claims.

What is claimed is:

1. One or more non-transitory computer storage media storing computer-useable instructions that, when used by one or more computing devices, cause the one or more computing devices to perform a method for displaying and recording patient physiologic data, the method comprising:
receiving signals from leads in near real-time, the signals corresponding to physiological measurements captured by the leads, the leads being associated with a patient;
generating waveforms in near real-time from the signals received, the waveforms representing the signals that correspond to the physiological measurements;
displaying, in near real-time via a graphical user interface (GUI), the waveforms as the waveforms are being generated;
receiving a manipulation of a time period associated with the GUI displaying the waveforms;
detecting an event for each instance when at least one of the waveforms being displayed via the GUI indicates that a corresponding signal received from one of the leads meets or exceeds a threshold for a physiological measurement;
for each event detected, automatically recording the at least one of the waveforms displayed in the GUI, the at least one waveform corresponding to the event detected, wherein the at least one waveform corresponding to the event is automatically recorded by temporarily holding the at least one waveform in a temporary queue for a configurable period of time; and
subsequent to said configurable period of time lapsing, automatically deleting a remaining portion of the automatically recorded at least one waveform from the temporary queue, the remaining portion consisting of the automatically recorded at least one waveform less the specific portion saved to the electronic medical record.

2. The media of claim 1, further comprising:
displaying a list of the events detected in the GUI and automatically recorded, where each event is individually selectable in the GUI; and
in response to selection of one of the events detected and displayed in the list in the GUI, displaying the automatically recorded at least one waveform corresponding to the event for playback of the at least one waveform in the GUI.

3. The media of claim 1, further comprising:
receiving an indication, via the GUI, to save a specific portion of the automatically recorded at least one waveform displayed for playback to an electronic medical record associated with the patient, wherein the specific portion of the at least one waveform is less than a whole of the at least one waveform; and
storing the specific portion of the automatically recorded at least one waveform to an electronic medical record associated with the patient.

4. The media of claim 1, wherein the manipulation of a time period associated with the GUI display includes playing, panning, scrolling, pausing, rewinding, or fast-forwarding the waveforms display.

5. The media of claim 1, further comprising receiving an indication from a clinician to display via the GUI the at least one waveform automatically recorded as corresponding to the event detected.

6. The media of claim 1, further comprising receiving an event measurement associated with the at least one waveform automatically recorded as corresponding to the event detected.

7. The media of claim 1, further comprising receiving an annotation associated with at least one waveform automatically recorded as corresponding to the event detected.

8. The media of claim 1, further comprising receiving an indication to drag physiologic data to a section of the GUI in order to generate a display of said physiologic data as additional waveforms in the section of the GUI.

9. The media of claim 1, further comprising receiving an indication to drag a particular waveform to a section of the GUI in order to generate a display of said waveforms as additional physiologic data in the section of the GUI.

10. One or more non-transitory computer storage media storing computer-useable instructions that, when used by one or more computing devices, cause the one or more computing devices to perform a method for displaying real-time and historical patient physiologic data, the method comprising:
receiving signals from leads corresponding to physiological measurements captured by the leads, the leads being associated with a patient;
generating a plurality of waveforms in near real-time from the signals received, the plurality of waveforms representing the signals that correspond to the physiological measurements;
displaying, in near real-time via a graphical user interface (GUI), the plurality of waveforms as the waveforms are being generated and physiologic data representing each of the signals received;
detecting an event for each instance when at least one of the plurality waveforms being displayed via the GUI indicates that a corresponding signal received from one of the leads meets or exceeds a threshold for a physiological measurement captured by the leads;
in response to each event detected, automatically saving the at least one of the plurality of waveforms as displayed in the GUI, the at least one waveform corresponding to the event detected, wherein the at least one waveform is automatically saved in a temporary queue for a configurable period of time;
displaying a list of the events detected in the GUI and automatically saved to the temporary queue, where each event is individually selectable in the GUI;
in response to selection of one of the events detected and displayed in the list in the GUI, displaying the automatically saved at least one waveform corresponding to the event for playback of the at least one waveform in the GUI;
receiving an event measurement of the at least one waveform during playback in the GUI, the at least one waveform corresponding to the event detected and selected from the list displayed in the GUI;
receiving an annotation for the at least one waveform during playback in the GUI, the at least one waveform corresponding to the event detected and selected from the list displayed in the GUI;
storing the automatically saved at least one waveform and the annotation for the automatically recorded at least one waveform to an electronic medical record associated with the patient; and
upon said configurable period of time lapsing, automatically purging the automatically saved at least one waveform and physiologic data that corresponds to the event detected for the automatically recorded at least one waveform from the temporary queue.

11. The media of claim 10, further comprising receiving an adjustment of time associated with the GUI.

12. The media of claim 11, wherein the adjustment of time includes playing, panning, scrolling, rewinding, fast forwarding, pausing, or any combination thereof.

13. The media of claim 10, further comprising receiving an indication from a clinician to electronically sign a selected view of the at least one waveform automatically saved for the event detected.

14. The media of claim 10, further comprising displaying, via the GUI, the at least one waveform that is saved in the electronic medical record in a historical queue.

15. A computer system for displaying real-time and historical patient physiologic data, the computer system comprising a processor coupled to a computer-storage medium, the computer-storage medium having stored thereon a plurality of computer software components executable by the processor, the computer software components comprising:
a signal component communicatively coupled to one or more leads, the signal component receiving signals from the one or more leads in near real-time, the signals corresponding to physiological measurements captured by the one or more leads, the one or more leads being associated with a patient;
a near real-time display component communicatively coupled to a display device, the near real-time display component generating waveforms in near real-time from the signals received, the waveforms representing the signals that correspond to the physiological measurements, wherein the near real-time display component displays, in near real-time via a graphical user interface (GUI), the waveforms as the waveforms are being generated;
an event component that automatically detects an event for each instance when at least one of the waveforms being displayed via the GUI indicates that a corresponding signal received from one of the leads meets or exceeds a threshold for a physiological measurement; and
a temporary queue component communicatively coupled to a first memory that, for each event detected, automatically records the at least one waveform as displayed in the GUI, the at least one waveform corresponding to the event detected, wherein the at least one waveform corresponding to the event is automatically recorded by temporarily holding the at least one waveform in a temporary queue for a configurable period of time and, subsequent to said configurable period of time lapsing, automatically deletes a remaining portion of the automatically recorded at least one waveform from the temporary queue, the remaining portion consisting of the automatically recorded at least one waveform less a specific portion saved to an electronic medical record.

16. The computer system of claim 15, further comprising a time manipulation component for receiving a manipulation of a time period associated with the GUI of the waveforms and, in response to the manipulation, manipulating the display of the waveforms in the GUI to playback, pan, scroll, pause, rewind, or fast-forward the waveforms.

17. The computer system of claim 15, further comprising an annotation and measurement component for receiving input and measuring characteristics relevant to the event detected.

18. The computer system of claim 15, further comprising a measurement component for measuring characteristics of the at least one waveform corresponding to the event detected.

19. The computer system of claim 15, further comprising a signing component in communication with the permanent save component, the signing component receiving an indication that a clinician has signed a record corresponding to the event detected.

20. The computer system of claim 15, further comprising a permanent save component that, upon the near real-time display component receiving an indication, via the GUI, to save the specific portion of the automatically recorded at least one waveform held in the temporary queue to the electronic medical record associated with the patient, stores the specific portion of the automatically recorded at least one waveform to the electronic medical record associated with the patient, wherein the specific portion of the at least one waveform is less than a whole of the at least one waveform.
